(19) United States
(12) Patent Application Publication
(10) Pub. No.: US 2023/0389841 A1
(43) Pub. Date: Dec. 7, 2023

(54) METHOD FOR PROVIDING SOLUTION BY CLASSIFYING EMOTIONAL STATE

(71) Applicants: Sang Cheul NAM, Incheon (KR); Myeong Sook KIM, Incheon (KR)

(72) Inventors: Sang Cheul NAM, Incheon (KR); Myeong Sook KIM, Incheon (KR)

(21) Appl. No.: 18/247,306

(22) PCT Filed: Mar. 29, 2022
(86) PCT No.: PCT/KR2022/095067

§ 371 Date: Mar. 30, 2023

(30) Foreign Application Priority Data
Mar. 30, 2021 (KR) 10-2021-0041297

(51) Int. Cl. A61B 5/16; A61B 5/00; G16H 20/70; G16H 10/20

(52) U.S. Cl. CPC A61B 5/165; A61B 5/7475; A61B 5/742; G16H 10/20; G16H 20/70

(57) ABSTRACT

A mode for carrying out the present invention may comprise: an emotion list displaying step in which a user terminal displays an emotion list; a user emotion selecting step in which the user terminal receives a plurality of selections of emotions felt by a user, as user emotions, from among the emotion list; a user emotion arranging step in which the user terminal arranges the selected plurality of user emotions; a specific emotional state information inputting step in which the user terminal receives inputs of specific emotional state information in the order of the arranged user emotions, and transmits same to an emotional state solution server; and a user emotion note generating step in which the emotional state solution server generates a user emotion note on the basis of the received specific emotional state information, and provides same to the user terminal.

### TECHNICAL FIELD

The present invention relates to a method of providing a solution by classifying an emotional state, and to a method of classifying a user's emotional state and providing a solution through the classified user's emotional state.

### BACKGROUND ART

Emotions (feelings) are feelings or moods that arise in the mind when encountering a certain phenomenon or event, and are caused by various causes such as physiological, physical, psychological, social, and cultural causes. Further, emotions may be classified from various perspectives into joy, anger, sorrow, pleasure, etc.

In this way, “emotions” may be caused by various causes and expressed in various forms, but in the past, there was a problem in that users did not receive appropriate solutions because there was no means to understand their emotions.

Therefore, there is an urgent need for a service means through which a user can receive a solution that the user wants and that meets the user's emotion.

### DISCLOSURE

### Technical Problem

The present invention is directed to providing a method of classifying a user's emotional state and providing a solution through the classified user's emotional state.

### Technical Solution

One aspect of the present invention provides a method of providing a solution by classifying an emotional state, which includes an emotion list displaying process in which a user terminal displays an emotion list; a user emotion selecting process in which, on the user terminal, a plurality of emotions felt by a user are selected as user emotions from among emotions in the emotion list; a user emotion arranging process in which the user terminal arranges the plurality of selected user emotions; a specific emotional state information inputting process in which the user terminal receives specific emotional state information in an order of the arranged user emotions to transmit the received specific emotional state information to an emotional state solution server; and a user emotion note generating process in which the emotional state solution server generates a user emotion note on the basis of the received specific emotional state information to provide the generated user emotion note to the user terminal.

The method may further include, after the user emotion note generating process is performed, a user emotion note providing process in which the emotional state solution server provides the user emotion note to a mentor terminal used by a mentor registered as a user's mentor; a user emotion note displaying process in which the mentor terminal displays the user emotion note received from the emotional state solution server; a mentoring comment inputting process in which the mentor terminal receives a mentoring comment input by the user's mentor to provide the received mentoring comment to the emotional state solution server; a mentoring comment providing process in which the emotional state solution server provides the mentoring comment received from the mentor terminal to the user terminal; and a mentoring comment displaying process in which the user terminal displays the mentoring comment received from the emotional state solution server.

The user emotion arranging process may include a primary arranging process in which the user terminal randomly and primarily arranges the plurality of selected user emotions;

and a final arranging process in which the user terminal finally arranges the user emotions after the user adjusts an arrangement order of the user emotions.

In the specific emotional state information inputting process, multiple-choice questions for specific emotional state information related to the user emotions may be displayed, and the user may select user emotions from among the multiple-choice questions for the specific emotional state information so that the specific emotional state information may be input.

In the specific emotional state information inputting process, an emotion card in which the user emotion is imaged may be displayed, and the specific emotional state information may be input in a question-and-answer format.

The emotion card may be displayed on an entirety of a screen of the user terminal.

### ADVANTAGEOUS EFFECTS

According to an embodiment of the present invention, an emotional state of a user is classified and a solution is provided through the classified emotional state, and thus the user can receive sympathy and advice from a mentor according to the user's emotional state.

Further, according to an embodiment of the present invention, emotions are classified into “emotions indicating the recovery of balance” and “emotions indicating the loss of balance,” a user selects emotions from among the classified emotions, and thus the emotions can be classified based on the Yin-Yang idea, that is, balance, which is the principle of all things, rather than good or bad emotions.

### DESCRIPTION OF DRAWINGS

FIG. 1 is a configuration diagram of a system for providing a solution by classifying an emotional state according to an embodiment of the present invention.

FIG. 2 is a flowchart illustrating a method of providing a solution by classifying an emotional state according to an embodiment of the present invention.

FIGS. 3 to 9 show diagrams of examples of screens of user terminals, on which solutions are provided by classifying an emotional state, according to an embodiment of the present invention.

FIGS. 10 and 11 show diagrams of examples of screens on which emotion cards are displayed according to an embodiment of the present invention.

### Best Mode Of The Invention

Hereinafter, embodiments of the present invention will be described with reference to the accompanying drawings. In assigning reference numerals to components of each drawing, it should be noted that the same components assigned the same numerals as much as possible even when the same components are illustrated in different drawings. In addition, in descriptions of the present invention, when detailed descriptions of related known configurations or functions are deemed to unnecessarily obscure the gist of the present invention, they will be omitted. In addition, embodiments of the present invention will be described below, but the technical idea of the present invention is not limited or restricted thereto and can be modified and implemented in various ways by those skilled in the art.

### Modes of the Invention

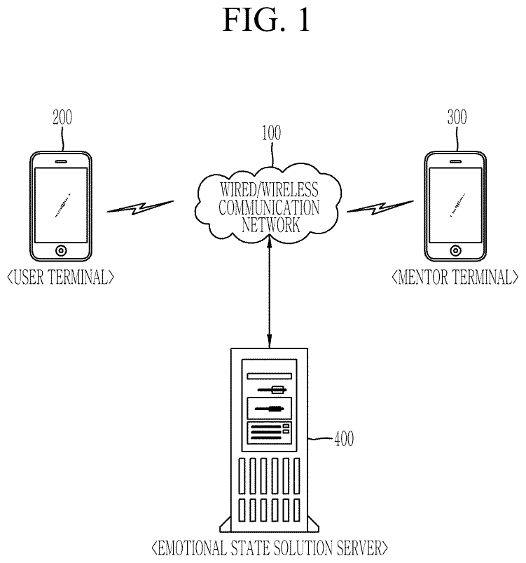

FIG. 1 is a configuration diagram of a system for providing a solution by classifying an emotional state according to an embodiment of the present invention.

The system for providing the solution by classifying the emotional state of the present invention may include a wired/wireless communication network 100, a user terminal 200, a mentor terminal 300, and an emotional state solution server 400.

The wired/wireless communication network 100 is a communication network that provides wired or wireless communication between the user terminal 200, the mentor terminal 300, and the emotional state solution server 400. When the wired/wireless communication network 100 is implemented as a wireless communication network, the wired/wireless communication network 100 may perform data communication using a wireless mobile communication network composed of a base transceiver station (BTS), a mobile switching center (MSC), and a home location register (HLR). Further, when the wired/wireless communication network 100 is implemented as a wired communication network, the wired/wireless communication network 100 may be implemented as a network (communication network) and may perform data communication according to an Internet protocol such as Transmission Control Protocol/Internet Protocol (TCP/IP) or the like.

The user terminal 200 is a terminal used by a user (mentee) who receives a solution according to an emotional state, and although in the drawings, an example of a smartphone is taken, the user terminal 200 may correspond to a desktop personal computer (PC), a tablet PC, a slate PC, a notebook computer, or the like, as well as the smartphone. Of course, the terminal to which the present invention can be applied is not limited to the above-described types, and may include any terminal that can communicate with external devices. This will also be applied to the mentor terminal 300.

The user terminal 200 displays an emotion list and the user selects a plurality of emotions felt by the user. Then, the user terminal 200 arranges the plurality of selected user emotions and receives specific emotional state information in the order of the arranged user emotions. Then, the user terminal 200 displays a user emotion note generated based on the received specific emotional state information. Further, the user terminal 200 receives and displays a mentoring comment provided by a mentor who has read the emotion note.

The mentor terminal 300 is a terminal used by the mentor who gives the mentoring comment to the user. The mentor terminal 300 displays the user emotion note received from the emotional state solution server 400, and receives the mentoring comment input by the mentor to provide the received mentoring comment to the emotional state solution server 400.

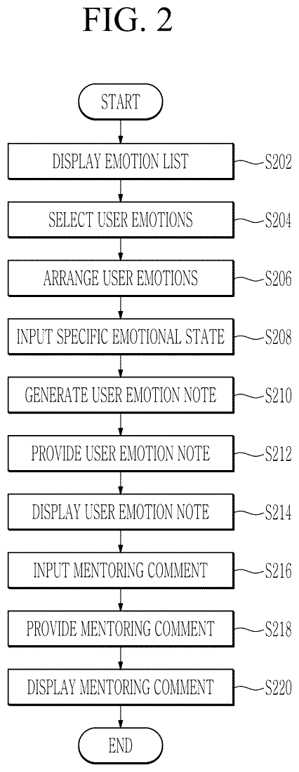

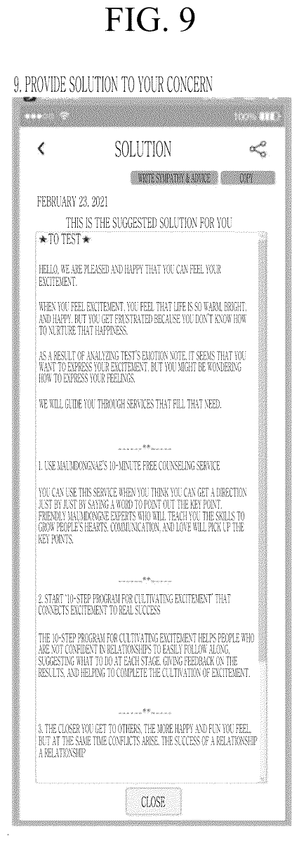

The emotional state solution server 400 has the same configuration as a normal web server in terms of hardware, and includes a program module that are implemented through various types of languages such as C, C++, Java, Visual Basic, and Visual C to perform various functions in terms of software. Further, the emotional state solution server 400 may be implemented using web server programs that are variously provided to general server hardware according to operating systems such as disk operating system (DOS), Windows, Linux, Unix, the Macintosh operating system, and the like. The emotional state solution server 400 provides the user emotion note to the mentor terminal 300 used by a mentor registered as the user's mentor, and the emotional state solution server 400 generates a user emotion note on the basis of the received specific emotional state information to provide the generated user emotion note to the user terminal 200. Further, the emotional state solution server 400 provides the mentoring comment received from the mentor terminal 300 to the user terminal 200. Hereinafter, a method of providing a solution by classifying such an emotional state will be described in detail with reference to FIGS. 2 to 11.

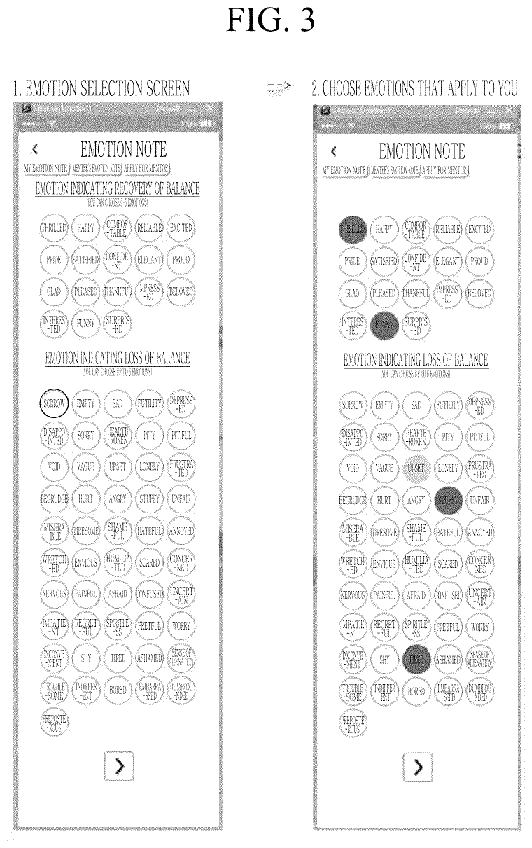

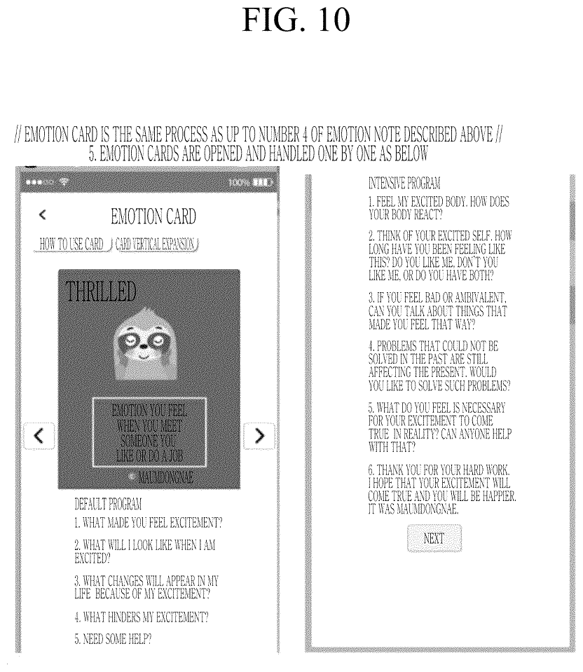

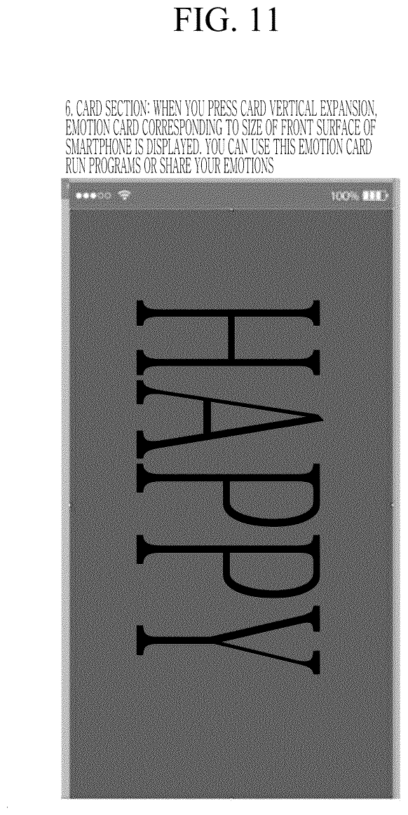

FIG. 2 is a flowchart illustrating a method of providing a solution by classifying an emotional state according to an embodiment of the present invention, FIGS. 3 to 9 show diagrams of examples of screens of user terminals, on which solutions are provided by classifying an emotional state according to the embodiment of the present invention, and FIGS. 10 and 11 show diagrams of examples of screens on which emotion cards are displayed according to the embodiment of the present invention.

As shown in FIG. 2, the method of providing the solution by classifying the emotional state of the present invention may include an emotion list displaying process S202 in which a user terminal 200 displays an emotion list, a user emotion selecting process S204 in which, on the user terminal 200, a plurality of emotions felt by a user are selected as user emotions from among emotions in the emotion list, a user emotion arranging process S208 in which the user terminal 200 arranges the plurality of selected user emotions, a specific emotional state information inputting process S208 in which the user terminal 200 receives specific emotional state information in the order of the arranged user emotions, and a user emotion note generating process S210 in which an emotional state solution server 400 generates a user emotion note on the basis of the received specific emotional state information to provide the generated user emotion note to the user terminal 200. Furthermore, the method may further include a user emotion note providing process S212 in which the emotional state solution server 400 provides the user emotion note to a mentor terminal 300 used by a mentor registered as a user's mentor, a user emotion note displaying process S214 in which the mentor terminal 300 displays the user emotion note received from the emotional state solution server 400, a mentoring comment inputting process S216 in which the mentor terminal 300 receives a mentoring comment input by the user's mentor to provide the input mentoring comment to the emotional state solution server 400, a mentoring comment providing process S218 in which the emotional state solution server 400 provides the mentoring comment received from the mentor terminal 300 to the user terminal 200, and a mentoring comment displaying process S220 in which the user terminal 200 displays the mentoring comment received from the emotional state solution server 400. Hereinafter, the above processes will be described in detail.

The emotion list displaying process S202 is a process in which the user terminal 200 displays the emotion list. An emotion list in which various emotion items are described is displayed as shown in FIG. 3.

The user emotion selecting process S204 is a process in which, on the user terminal 200, a plurality of emotions felt by a user are selected as user emotions from among emotions in the emotion list. That, as shown in FIG. 3, the user selects emotions corresponding to himself or herself. The emotions are classified into “emotions indicating the recovery of balance” and “emotions indicating loss of balance,” the user selects emotions from among the classified emotions, and thus the emotions may be classified based on the Yin-Yang (balance), which is the principle of all things, rather than good or bad emotions.

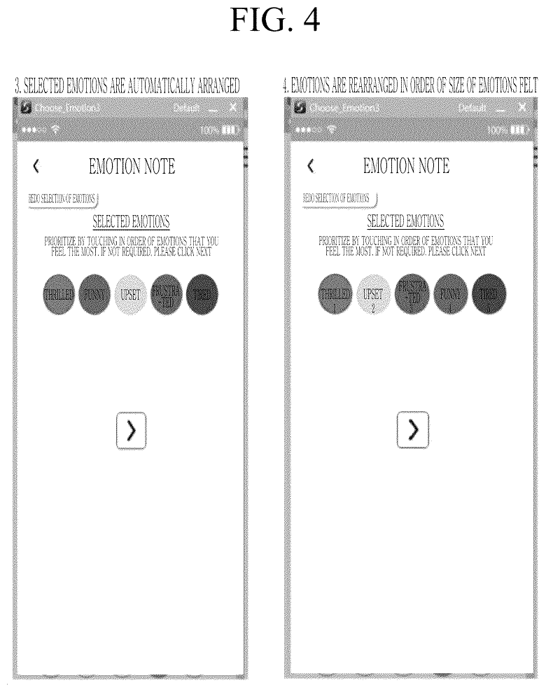

The user emotion arranging process S208 is a process in which the user terminal 200 arranges the plurality of selected user emotions. As shown in FIG. 4, the user emotion arranging process S208 may include a primary arranging process in which the user terminal 200 randomly and primarily arranges the plurality of selected user emotions, and a final arranging process in which the user terminal 200 finally arranges the user emotions after the user adjusts an arrangement order of the user emotions. Therefore, the user may arrange the emotions in order of priority by touching the emotions in the order of the emotions the user feels the most.

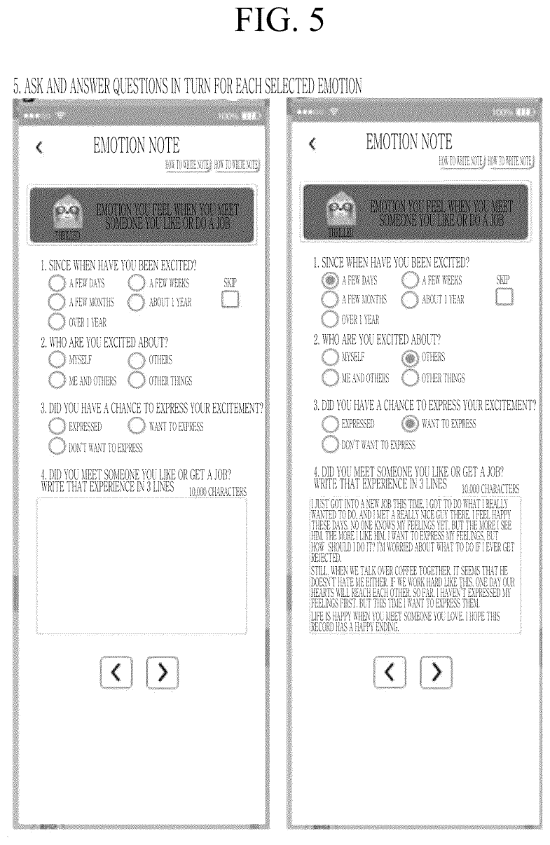

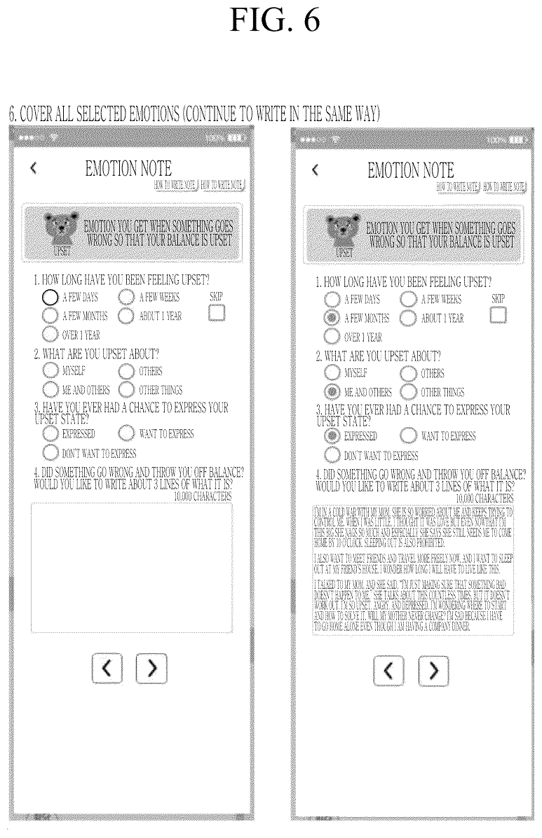

The specific emotional state information inputting process S208 is a process in which the user terminal 200 receives the specific emotional state information in the order of the arranged user emotions. In the specific emotional state information inputting process S208, multiple-choice questions for the specific emotional state information related to the user emotions may be displayed, and the user may select user emotions from among the multiple-choice questions for the specific emotional state information so that the specific emotional state information may be input. For example, as shown in FIGS. 5 and 6, the specific emotional state is input for each emotion felt by the user in a multiple choice format.

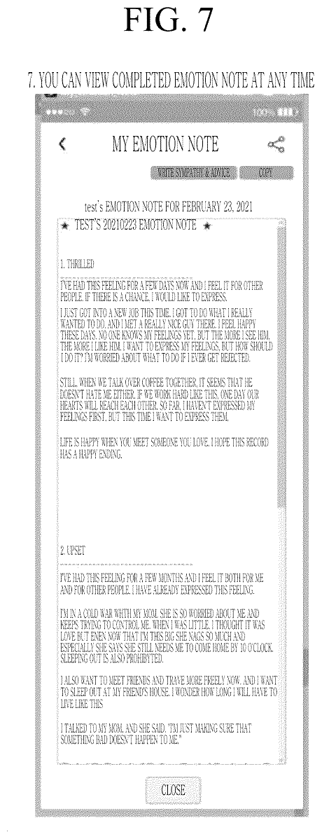

The user emotion note generating process S210 is a process in which the emotional state solution server 400 generates and stores a user emotion note on the basis of the received specific emotional state information and provides the generated and stored user emotion note to the user terminal 200. For example, when specific emotional state information on “exciting emotion” is input as shown in FIG. 5, specific information on the “exciting emotion” is organized to generate a user emotion note in phrases, so that a user emotion note may be generated and displayed on the user terminal 200 as shown in FIG. 7. For reference, the emotional state solution server 400 may write phrases of the emotion note on the basis of keywords of the specific emotional state information through pre-registered analysis and writing programs.

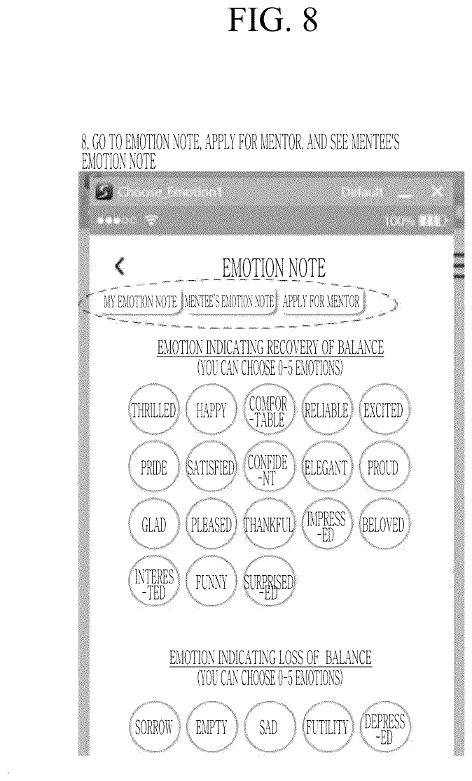

In the user emotion note providing process S212, the emotional state solution server 400 provides the user emotion note to the mentor terminal 300 used by the mentor registered as the user's mentor. For example, when “apply for a mentor” shown in FIG. 8 is pressed, the user emotion note is provided to the registered mentor. For reference, when the user becomes a mentor, the user can find and register a mentee, and the mentee may designate and apply for a mentor. The mentor and the mentee should give each other permission to be finally registered. The mentor may read an emotion note of the mentee and write and leave “sympathy and advice.”

The user emotion note displaying process S214 is a process in which the mentor terminal 300 displays the user emotion note received from the emotional state solution server 400. Therefore, the user's mentor may read the user emotion note and know the user's emotional state.

The mentoring comment inputting process S216 is a process in which the mentor terminal 300 receives a mentoring comment input by the user's mentor to provide the input mentoring comment to the emotional state solution server 400.

The mentoring comment providing process S218 is a process in which the emotional state solution server 400 provides the mentoring comment received from the mentor terminal 300 to the user terminal 200.

The mentoring comment displaying process S220 is a process in which the user terminal 200 displays the mentoring comment received from the emotional state solution server 400. Therefore, the user may have mental healing with the mentoring comment of sympathy and advice provided by the user's mentor.

Meanwhile, in the specific emotional state information inputting process S208, as shown in FIGS. 5 and 6, multiple-choice questions for the specific emotional state information related to the user emotions may be displayed, and the user may select user emotions from among the multiple-choice questions for the specific emotional state information so that the specific emotional state information may be input. In addition, as another embodiment, the specific emotional state information may be input as an emotion card method.

In inputting the specific emotional state information, as shown in FIG. 10, an emotion card obtained by imaging the user emotion is displayed, and the specific emotional state information is input in a question-and-answer format.

The emotion card may be displayed on the entire screen of the user terminal 200. Therefore, when the user selects the vertical expansion of the card, an emotion card corresponding to the size of a front surface of a smartphone is displayed. The user may use the emotion card to run programs or share the emotions. For reference, in addition to the emotion card, all desires and needs cards, feeling cards, career cards, and the like may be used in the same way as the emotion card.

Meanwhile, as described above, optimal embodiments have been disclosed in the accompanying drawings and specifications. Although specific terms have been used herein, they are only used for the purpose of describing the present invention and are not used to limit the scope of the present invention described in the claims. The same process can work for desires (needs) and feelings as well as emotions.

Therefore, it will be understood by those skilled in the art that various modifications and equivalent other examples may be made. Therefore, the scope of the present invention should be defined by the technical scope of the present invention of the appended claims.

1. A method of providing a solution by classifying an emotional state, comprising:
an emotion list displaying process in which a user terminal displays an emotion list;
a user emotion selecting process in which, on the user terminal, a plurality of emotions felt by a user are selected as user emotions from among emotions in the emotion list;
a user emotion arranging process in which the user terminal arranges the plurality of selected user emotions;
a specific emotional state information inputting process in which the user terminal receives specific emotional state information in an order of the arranged user emotions to transmit the received specific emotional state information to an emotional state solution server; and
a user emotion note generating process in which the emotional state solution server generates a user emotion note on the basis of the received specific emotional state information to provide the generated user emotion note to the user terminal.

2. The method of claim 1, further comprising, after the user emotion note generating process is performed:
a user emotion note providing process in which the emotional state solution server provides the user emotion note to a mentor terminal used by a mentor registered as a user's mentor;
a user emotion note displaying process in which the mentor terminal displays the user emotion note received from the emotional state solution server;
a mentoring comment inputting process in which the mentor terminal receives a mentoring comment input by the user's mentor to provide the received mentoring comment to the emotional state solution server;
a mentoring comment providing process in which the emotional state solution server provides the mentoring comment received from the mentor terminal to the user terminal; and
a mentoring comment displaying process in which the user terminal displays the mentoring comment received from the emotional state solution server.

3. The method of claim 1, wherein the user emotion arranging process includes:
a primary arranging process in which the user terminal randomly and primarily arranges the plurality of selected user emotions; and
a final arranging process in which the user terminal finally arranges the user emotions after the user adjusts an arrangement order of the user emotions.

4. The method of claim 1, wherein, in the specific emotional state information inputting process, multiple-choice questions for specific emotional state information related to the user emotions are displayed, and the user selects user emotions from among the multiple-choice questions for the specific emotional state information so that the specific emotional state information is input.

5. The method of claim 1, wherein, in the specific emotional state information inputting process, an emotion card in which the user emotion is imaged is displayed, and the specific emotional state information is input in a question-and-answer format.

6. The method of claim 5, wherein the emotion card is displayed on an entirety of a screen of the user terminal.
